(19) United States
(12) Patent Application Publication
(10) Pub. No.: US 2022/0079913 A1
(43) Pub. Date: Mar. 17, 2022

(54) DIHYDROMYRICETIN WITH ANTI-ALCOHOLIC EFFECT

(71) Applicant: Shanghai Spark Pharmaceutical Co., Ltd., Shanghai (CN)

(72) Inventors: KUNYUAN SONG, Shanghai (CN); WEIWEI CHEN, Shanghai (CN)

(21) Appl. No.: 17/290,939

(22) PCT Filed: Jun. 18, 2020
(86) PCT No.: PCT/CN2020/096795

§ 371 Date: May 3, 2021

(30) Foreign Application Priority Data
Apr. 29, 2020 (CN) 202010357439.5

(51) Int. Cl. A61K 31/352; A61P 1/16; A61P 25/32; A61P 39/02; A23L 33/105; A23L 33/00

(52) U.S. Cl. CPC A61K 31/352; A61P 1/16; A61P 25/32; A23V 2002/00; A23L 33/105; A23L 33/40; A61P 39/02

(57) ABSTRACT

This application discloses a new use of dihydromyricetin, that is, its application in preparing foods, health products or medicines with anti-alcoholic effect. Its advantages are: dihydromyricetin has a good function of dispeling the effects of alcohol. Dihydromyricetin can increase the expression of GABAARs a4 subgroup in the hippocampus and neurons, and play a role in resisting alcoholism and alcohol dependence. Dihydromyricetin can also effectively prevent the depletion of reduced glutathione and increase of malondialdehyde in the liver caused by alcohol, as well as reducing triglyceride content, reducing the degree of fatty degeneration of liver cells, and has a better effect of preventing and treating alcoholic liver injury.

### TECHNICAL FIELD

This application relates to the field of medical technology, in particular to a flavonoid compound with anti-alcoholic effect, namely dihydromyricetin.

### BACKGROUND OF RELATED ARTS

Drinking culture, as a part of our country's social culture, has long penetrated into the daily lives of many people. Drinking alcohol in moderation can promote blood circulation, relieve dampness and relieve pain, and relax the mood. However, excessive drinking can easily lead to drunkenness or acute alcoholism, which greatly harms health. Acute alcoholism caused by heavy drinking has become one of the diseases with the highest incidence during holidays. The development of safe and efficient hangover products has attracted more and more attention from the society.

Dihydromyricetin, molecular formula: C₁5H₁₂O₈, relative molecular weight: 320.25, chemical structure: (2R,3R)-3,5,7-trihydroxy-2-(3,4,5-trihydroxyphenyl) benzodihydropyran-4-one. Dihydromyricetin is a white needle-like crystal with a melting point of 245˜246° C. Low solubility in room temperature and cold water, easily soluble in methanol, ethanol and acetone, very slightly soluble in ethyl acetate, and hardly soluble in chloroform and petroleum ether. Dihydromyricetin can be prepared by mixing the raw materials of rattan tea with a certain concentration of ethanol in proportion, filtering, and decoloring, concentrating, and crystallization of the extract, drying, crushing, and storing in aluminum bag packaging.

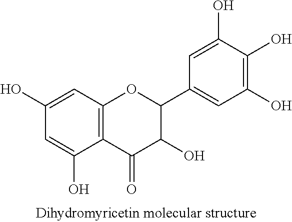 [SMILES: O=C1c2c(O)cc(O)cc2OC(c2cc(O)c(O)c(O)c2)C1O]

Dihydromyricetin is widely present in plants of the genus Snake grapes of the Snake grape family, as well as in plants of the Myricaceae, Rhododendronaceae, Garciniaceae, Euphorbiaceae, Olivaceae, Leguminosae, Salicaceae and Salicaceae. Previous studies have confirmed that dihydromyricetin has various pharmacological effects such as anti-oxidation, anti-tumor, anti-pathogenic microorganisms and regulating blood lipids.

### SUMMARY

This application provides a new use of flavonoids, especially dihydromyricetin.

This application adopts the following technical solutions:

An application of flavonoids in the preparation of foods, health products or medicines with anti-alcoholic effect.

The anti-alcohol effect is the resistance of alcohol-dependent effects, prevention or treatment of alcoholic liver injury, shortening a sobering time, and increasing tolerance to alcohol.

The flavonoid compound is dihydromyricetin. The dosage of dihydromyricetin is 1˜5000 mg/kg. Further, it is 350 to 1050 mg/kg.

Dihydromyricetin has a good anti-alcoholic effect, can increase the expression of GABAARs a4 subgroup in hippocampus and neurons, and play a role in resisting alcoholism and alcohol dependence. Dihydromyricetin can also effectively prevent liver reduction due to alcohol. Glutathione depletion and malondialdehyde increase, reduce triglyceride content, reduce the degree of fatty degeneration of liver cells, and have a better effect of preventing and treating alcoholic liver injury.

### DETAILED DESCRIPTIONS OF EMBODIMENTS

In order to make the purpose, technical solutions and advantages of the present application clearer, the technical solutions of the present application will be described clearly and completely in conjunction with specific embodiments of the present application. Obviously, the described embodiments are only a part of the embodiments of the present application, rather than all the embodiments.

Based on the embodiments in this application, all other embodiments obtained by those of ordinary skill in the art without creative work shall fall within the protection scope of this application.

### Example 1: Safety Test

1. Test Purpose

### Observation of the Acute Toxicity of Dihydromyricetin on ICR Mice

2. Reagents and Materials Related Information

Test substance: Dihydromyricetin

Storage condition of test substance: 4° C., dry and dark

Other reagents: sterile water for injection

Source: Guangdong Aixida Pharmaceutical Co., Ltd.

Properties: colorless transparent liquid

Specification: 500 ml/bottle

Storage conditions: RT

3. Drug Preparation

Solvent: sterile water for injection

Preparation of test substance: weighing the test substance of the required quality; adding sterile water for injection to prepare it to the required concentration; mixing well before administration.

4. Laboratory Animal Breeding

Animal breeds and strains: ICR mice

Level: SPF level

Gender: half male and half female

Source: Shanghai Slake Experimental Animal Co., Ltd.

Laboratory animal quality certificate number: 20170005014935 Laboratory animal production license number: SCXK (Shanghai) 2017-0005

Number of animals: ordering 15 males and 15 females each, a total of 30 used for experiment

Animal age at the beginning of the experiment: 6-10 w

Animal weight at the beginning of the experiment: 20 g±20%

Adaptation time: 4 days, the same feeding conditions as the experiment

Animal numbering method: equipping each squirrel cage with an identification card with information such as experiment number, experiment group, experimenter's name, animal breed and gender; marking the mouse with a line at the base of the tail.

Environment: Keeping the animal room at a temperature of 23±2° C., a humidity of 40-70%, and alternating light and dark for 12 hours, raising five animals in each cage, and changing the bedding twice a week (corncob bedding, Suzhou Daichuan Trading Co., Ltd.).

Food and drinking water: feeding SPF rats Growth and reproduction feed Co60 sterilization during the adaptation period, purchased from Beijing Co-operative Feeds Co; using autoclaved filtered water as water for experimental animals.

Animal selection and fasting: remaining the animals used in the experiment healthy; making the animals eat and drink freely during the experiment.

5. Test Method

After the adaptation period, 30 experimental animals were divided into 3 groups, half male and half male. After fasting for 12 hours, according to the body weight, the control reagents or the test substance were administered orally respectively. The administration situation is shown in Table 1. The animal's clinical symptoms after administration are observed, with abnormal records. The animals were weighed before the administration and on the 1, 3, 5, and 7 days after the administration. At the end of the experiment, the animals were euthanized by inhalation of excessive CO₂

TABLE 1
the administration condition of acute toxicity test
substance and control reagent

| | | Number | | | | |
|---|---|---|---|---|---|---|
| | | of | | | | |
| | | animals | | | Dosing | |
| | | (5 males | | Dosing | concen- | Way of |
| | | and 5 | Dose | volume | tration | adminis- |
| Group | Test substance | males) | (mg/kg) | (ml/kg) | (mg/ml) | tration |
| 1 | Vehicle (water) | 10 | — | 20 | — | i.g. once |
| 2 | Dihydromyricetin | 10 | 3500 | 20 | 175 | i.g. once |
| 3 | Dihydromyricetin | 10 | 5000 | 20 | 250 | i.g. once |

Test Results

1) Mortality Rate

During the test period, the animals showed no obvious abnormality with the naked eye, and the mortality rate was 0, as shown in Table 2.

TABLE 2
effect of single oral administration of the test
substance (dihydromyricetin) on the mortality

| | Mortality rate (%)/day | | | | | | | |
|---|---|---|---|---|---|---|---|---|
| Test substance | 0 | 1 | 2 | 3 | 4 | 5 | 6 | 7 |
| G1 Vehicle (water) | 0 | 0 | 0 | 0 | 0 | 0 | 0 | 0 |
| G2 Dihydromyricetin | 0 | 0 | 0 | 0 | 0 | 0 | 0 | 0 |
| 3500 mg/kg | | | | | | | | |
| G3 Dihydromyricetin | 0 | 0 | 0 | 0 | 0 | 0 | 0 | 0 |
| 5000 mg/kg | | | | | | | | |

2) Body Weight

The body weight of the mice in Vehicle group was from 22.38±0.66 g at the beginning of the experiment to 27.43±1.31 g at the end of the experiment. The body weight steadily increased during the experiment. The body weight of animals in the dihydromyricetin 3500 mg/kg and dihydromyricetin 5000 mg/kg groups increased steadily, and compared with the vehicle group, there was no statistically significant difference (p>0.05), see Table 3.

TABLE 3
effect of single oral administration of test substance (dihydromyricetin)
on the body weight (g) of mice (n = 10,)

| | body weight (g)/day | | | | |
|---|---|---|---|---|---|
| Test substance | 0 | 1 | 3 | 5 | 7 |
| G1 Vehicle(water) | 22.38 ± 0.66 | 24.79 ± 0.90 | 26.01 ± 1.02 | 26.74 ± 1.18 | 27.43 ± 1.31 |
| G2 Dihydromyricetin | 21.82 ± 0.48 | 24.82 ± 0.94 | 25.99 ± 0.92 | 26.73 ± 1.00 | 27.43 ± 1.08 |
| 3500 mg/kg | | | | | |
| G3 Dihydromyricetin | 21.40 ± 0.11 | 23.07 ± 0.38 | 24.43 ± 0.41 | 25.01 ± 0.47 | 26.02 ± 0.57 |
| 5000 mg/kg | | | | | |

Conclusion

After a single oral gavage of the test substance (dihydromyricetin) 3500 mg/kg and 5000 mg/kg, the animal has no visible abnormality with the naked eye, and the weight steadily increases, so the MTD value of the test substance is 5000 mg/kg.

### Example 2: Effectiveness Test

1. Test Purpose

Verification whether dihydromyricetin can effectively exert the anti-alcoholic effect

2. Information about Reagents and Materials

Test substance: Dihydromyricetin

Storage condition of test substance: 4° C., dry and dark

Other reagents:

1) Sterilized Water for Injection

Source: Guangdong Aixida Pharmaceutical Co., Ltd.

Properties: colorless transparent liquid

Specification: 500 ml/bottle

Storage conditions: RT

2) Wine

Brand: Red Star Erguotou

Specification: 500 ml/bottle

Storage conditions: RT

3. Drug Preparation

Solvent: sterile water for injection

Preparation of test substance: weighing the test substance of the required quality; adding sterile water for injection to prepare it to the required concentration; mixing well before administration.

4. Laboratory Animal Breeding

Animal breeds and strains: ICR mice

Level: SPF level

Gender: half male and half female

Source: Shanghai Slake Experimental Animal Co., Ltd.

Laboratory animal quality certificate number: 20170005014935 Laboratory animal production license number: SCXK (Shanghai) 2017-0005

Number of animals: ordering 50 males and 50 females each, a total of 100 used for experiment

Animal age at the beginning of the experiment: 6-10 w

Animal weight at the beginning of the experiment: 20 g±20%

Adaptation time: 4 days, the same feeding conditions as the experiment

Animal numbering method: equipping each squirrel cage with an identification card with information such as experiment number, experiment group, experimenter's name, animal breed and gender; marking the mouse with a line at the base of the tail.

Environment: Keeping the animal room at a temperature of 23±2° C., a humidity of 40˜70%, and alternating light and dark for 12 hours, raising five animals in each cage, and changing the bedding twice a week (corncob bedding, Suzhou Daichuan Trading Co., Ltd.).

Food and drinking water: feeding SPF rats Growth and reproduction feed Co60 sterilization during the adaptation period, purchased from Beijing Co-operative Feeds Co; using autoclaved filtered water as water for experimental animals.

Animal selection and fasting: remaining the animals used in the experiment healthy; making the animals eat and drink freely during the experiment.

5. Test Method

1) Test 1

After the adaptation period, 40 experimental animals were divided into 4 groups, half male and half male. After fasting for 12 hours, according to the body weight, the test substance was administered orally respectively. The drunkenness rate (disappearance of the righting reflex) and mortality of the mice are observed, and the amount and time of gavage required for the mice with the disappearance of the righting reflex without death were found out. Table 4 shows the drunkenness condition.

TABLE 4
The drunkenness test condition

| | | Number of | | |
|---|---|---|---|---|
| | | animals | | |
| | | (5 males and 5 | Dose | Way of |
| Group | Test substance | males) | (ml/kg) | administration |
| 1 | Wine (Red Star | 10 | 20 | i.g. once |
| | Erguotou) | | | |
| 2 | Wine (Red Star | 10 | 18 | i.g. once |
| | Erguotou) | | | |
| 3 | Wine (Red Star | 10 | 16 | i.g. once |
| | Erguotou) | | | |
| 4 | Wine (Red Star | 10 | 14 | i.g. once |
| | Erguotou) | | | |

Test Results

As shown in Table 5, in the Group 2 of animals at a dose of 18 ml/kg, the drunkenness rate of the animals was 100%, and the mortality rate of the animals was 0%, which is suitable for the test of the dose of anti-alcoholic effect.

TABLE 5
Drunkenness test results

| | Dose of | | | | | | | |
|---|---|---|---|---|---|---|---|---|
| | test | Number | Number | Number | | | Tolerance | Sobering |
| | substance | of | of | of | mortality | Drunkenness | time | time |
| Group | ml/kg | animals | deaths | drunks | rate % | rate % | min | min |
| 1 | 20 | 10 | 5 | 10 | 50 | 100 | 32.70 | 567.30 |
| 2 | 18 | 10 | 0 | 10 | 0 | 100 | 42.50 | 373.40 |
| 3 | 16 | 10 | 0 | 6 | 0 | 60 | 271.10 | 141.40 |
| 4 | 14 | 10 | 0 | 2 | 0 | 20 | 493.60 | 30.70 |

2) Test 2

60 experimental animals were divided into 3 groups, half male and half male. After fasting for 12 hours, according to the body weight, the control reagents the test substance were administered orally respectively. As shown in Table 6, the sobering time (the time for the disappearance of the righting reflex), the drunken rate and the mortality of the mice after 10 hours of drinking were observed.

TABLE 6
the administration of test substances
and control reagents in the hangover test

| | | Number of | | |
|---|---|---|---|---|
| | | animals (5 | | |
| | Test | males and 5 | Dose | Way of |
| Group | substance | males) | mg/kg | administration |
| 1 | Water | 10 | — | i.g. once |
| 2 | Dihydromyricetin | 10 | 350 | i.g. once |
| 3 | Dihydromyricetin | 10 | 1050 | i.g. once |

The test results are shown in Table 7. After 10 hours of drinking, the sobering time (214 min) of the group with the dose of 1050 mg/kg dihydromyricetin was significantly lower than that of the control group (324 min), which was statistically different. Therefore, dihydromyricetin has a good anti-alcoholic effect.

In the anti-alcoholic test, one mouse died in the group with the dose of 1050 mg/kg. The autopsy found that the gastrointestinal bleeding was severely fatal. It is speculated that the dead mouse may be a weakly diseased mouse, which is quite different from the 99 animals in the group, so this animal was eliminated data.

TABLE 7
the data of the anti-alcoholic test

| | | | Number | Number | Number | | | Tolerance | Sobering |
|---|---|---|---|---|---|---|---|---|---|
| | | Dose | of | of | of | mortality | Drunkenness | time | time |
| Group | Test substance | mg/kg | animals | deaths | drunks | rate % | rate % | min | min |
| 1 | Water | 20 | 20 | 0 | 18 | 0 | 90 | 105.60 | 324.55 |
| 2 | Dihydromyricetin | 350 | 20 | 0 | 19 | 0 | 95 | 77.40 | 376.35 |
| 3 | Dihydromyricetin | 1050 | 20 | 1 | 14 | 5 | 70 | 193.00 | 214.32 |

The above descriptions are only examples of the present application, and are not used to limit the present application. For those skilled in the art, this application can have various modifications and changes. Any modification, equivalent replacement, improvement, etc. made within the spirit and principle of this application shall be included in the scope of the claims of this application.

What is claimed:

1. An application of flavonoids in the preparation of foods, health products or medicines with anti-alcoholic effect.

2. The application according to claim 1, wherein the anti-alcoholic effect is to resist alcoholism.

3. The application according to claim 1, wherein the anti-alcoholic effect is the resistance of alcohol-dependent effects.

4. The application according to claim 1, wherein the anti-alcoholic effect is prevention or treatment of alcoholic liver injury.

5. The application according to claim 1, wherein the anti-alcoholic effect is shortening a sobering time.

6. The application according to claim 1, wherein the anti-alcohol effect is increasing tolerance to alcohol.

7. The application according to claim 1, wherein the flavonoid compound is dihydromyricetin.

8. The application according to claim 7, wherein the dose of dihydromyricetin is 1-5000 mg/kg.

9. The application according to claim 8, wherein the dose of dihydromyricetin is 350-1050 mg/kg.
